(19) United States
(12) Patent Application Publication
(10) Pub. No.: US 2018/0326170 A1
(43) Pub. Date: Nov. 15, 2018

(54) MEDICAL DEVICE SYSTEM INCLUDING A TRACHEAL TUBE AND METHOD

(71) Applicant: Brian Kamradt, Indianapolis, IN (US)

(72) Inventor: Brian Kamradt, Indianapolis, IN (US)

(21) Appl. No.: 15/972,817

(22) Filed: May 7, 2018

Related U.S. Application Data
(60) Provisional application No. 62/602,851, filed on May 9, 2017.

(51) Int. Cl. A61M 16/04; A61M 16/08; A61M 16/20

(52) U.S. Cl. CPC A61M 16/0465; A61M 16/0427; A61M 2205/0216; A61M 16/208; A61M 16/0816

(57) ABSTRACT

A method and apparatus for accomplishing placement and removal of an inner cannula with in an outer cannula endotracheal or other tracheal tube circuit for a critically ill patient without having to disconnect the patient from a respirator machine. An inner cannula is positioned in the outer cannula of an endotracheal or other tracheal tube through a wall of the respirator tubing set in a manner that maintains the integrity of the respiration system.

This application claims priority under 35 U.S.C. § 119 to U.S. Provisional App. No. 62/602,851, which was filed on May 9, 2017 and is expressly incorporated by reference.

### TECHNICAL FIELD

The present disclosure relates generally to medical device systems and methods, and, in particular, to tracheal devices including endotracheal devices and tracheostomy devices.

### BACKGROUND

Ventilation of patients by connecting them to a respirator machine is a widespread practice to aid in their breathing when critically ill, such as during or immediately following an operation. A typical respirator machine provides a breathing gas for the patient having a high level of oxygen in one tube and receives, back in another tube the gases exhausted by the lungs of the patient. These two tubes are joined together at a manifold position that is as close to the point of entry to the patient as possible. A single additional tube positioned into the trachea of the patient, either directly or through the nose or mouth, is connected to the manifold. A principal goal of such a ventilation technique is to maintain a high level of blood oxygenation in the patient without the heart and lungs having to work excessively hard.

A patient so connected to a respirator requires periodic replacement of the endotracheal or tracheostomy tube. The present technique as widely practiced in hospitals is to disconnect the respirator hoses from the patient, and then replace the endotracheal or tracheostomy tube. During this periodic process, some temporary breathing assistance is provided, but not of the quality or quantity as provided by the respirator. This interruption necessarily results in the oxygen level of the blood to decrease, and for the heart and lungs to have to work harder, a problem with many critically ill patients. Much has been written about the solution to this problem, typical suggestions being to hyperinflate the lungs before and/or after the replacement process. But this technique does not maintain the same level of breathing assistance as when the patient's connection to the respirator is uninterrupted.

The use of positive end expiratory pressure (PEEP) has gained wide popularity in the management of the respiratory status of critically ill patients. Generally, this known technique maintains through an appropriate respirator a slightly positive gaseous pressure to the patient at all times. The basic advantage of this technique is that it allows a lower concentration of oxygen to be provided to the patient in order to maintain an adequate level of blood oxygenation. It is thought that this is due to the fact that the positive pressure maintains a larger number of the patient's lung alveoli open during the respiratory support, thereby increasing the effective lung area of ventilation and decreasing ventilation/per fusion defects. Although there is some controversy as to the effect of interrupting such a positive pressure for various nursing maneuvers such as suctioning, changing tubing, etc., empirical data tends to suggest that the interruption of the positive pressure leads to an immediate effect in a sudden collapse of lung alveoli. This data also suggests that it takes a substantial amount of time after such an interruption for the positive pressure to restore the lung alveoli to their open state.

### SUMMARY

According to an aspect of the disclosure, a method and apparatus for placement and removal of an inner cannula with in an enclosed endotracheal or other tracheal tube circuit for a critically ill patient is disclosed. The method includes performing placement and removal of an inner cannula of a patient who is connected to a respirator machine without interrupting the connection of the patient to the respirator machine. An opening is provided in a wall in the fluid conduit between the patient and the respirator machine at a location close to entry into the patient. The opening is sealed in a manner to permit insertion and removal of the inner cannula therethrough without opening the respiratory supply system to the atmosphere.

According to another aspect, a medical device system is disclosed. The medical device system comprises a tracheal tube configured for insertion into an opening of a patient and a manifold removably coupled to the tracheal tube. The tracheal tube includes an outer cannula extending from a proximal end to a distal end configured to be positioned between in a patient's trachea. The outer cannula includes a lumen extending from a first opening defined in the proximal end to a second opening defined in the distal end. The tracheal tube also includes an inner cannula removably positioned in the lumen of the outer cannula. The inner cannula has a proximal end that extends outwardly from the first opening of the outer cannula, a distal end positioned adjacent to the distal end of the outer cannula, and a passageway extending through the distal and proximal ends.

The manifold of the medical device system is removably coupled to the proximal end of the outer cannula. The manifold includes an outlet connected to the first opening of the lumen of the outer cannula, a first inlet sized to receive and permit the passage of the inner cannula through the manifold into the first opening of the lumen of the outer cannula, and a second inlet configured to be coupled to an outlet of a respirator machine to permit airflow from the second inlet into the outlet. The medical device system also includes a cover positioned over the first inlet. The cover including at least one valve flap operable to be moved between a first position in which the first inlet is sealed and a second position in which the inner cannula is permitted to enter the first inlet.

In some embodiments, the first inlet may be sized to receive an endoscope sized to be inserted into the outer cannula. The first inlet may be sized to receive a tool configured to clean the outer cannula. Additionally, in some embodiments, the first inlet may be sized to receive other tools or instruments to be inserted into the outer cannula.

In some embodiments, the inner cannula may include a curved outer surface that extends between its distal end and its proximal end, and the at least one flap may be configured to engage the curved outer surface to create a seal between the inner cannula and the at least one valve flap when the inner cannula is positioned in the first inlet.

In some embodiments, the cover and the at least one valve flap may be formed from an elastomeric material. Additionally, in some embodiments, the elastomeric material may be at least one of rubber and silicone.

In some embodiments, the at least one valve flap may include a plurality of triangular valve flaps extending from an outer rim of the cover. The outer rim may be coupled to the manifold.

In some embodiments, the manifold may include a first housing and a second housing movable coupled to the first housing. Additionally, in some embodiments, the second housing may be movable between a first position in which the first inlet is aligned with the outlet and a second position in which the first inlet is positioned transverse to the outlet. In some embodiments, when the second housing is the second position, a section of the first housing may be positioned over the first inlet.

In some embodiments, the second inlet may be in fluid communication with the outlet when the second housing is in the second position. Additionally, in some embodiments, the second inlet may be positioned transverse to the outlet when the second housing is in the first position, and the second inlet may be aligned with the outlet when the second housing is in the second position.

In some embodiments, the medical device system may further comprise a connector having a first end sized to be positioned in the opening defined in the proximal end of the outer cannula and a second end sized to be positioned over the outlet of the manifold.

According to another aspect, a medical device system comprises a tracheal tube configured for insertion into an opening of a patient and a conduit configured to be coupled to a proximal end of the tracheal tube. The tracheal tube includes an outer cannula, and the conduit comprises a manifold including an outlet connected to the outer cannula, a first inlet sized to receive and permit the passage of an inner cannula through the manifold into the outer cannula, and a second inlet configured to be coupled to an outlet of a respirator machine to permit airflow from the second inlet into the endotracheal tube. The conduit also comprises a cover positioned over the first inlet. The cover includes at least one valve flap operable to selectively seal the first inlet.

In some embodiments, the inner cannula may be one of a plurality of inner cannulas is sized to be positioned in the outer cannula.

According to another aspect, a method of operating a medical device system is disclosed. The method comprises aligning an inner cannula with a first inlet of a manifold coupled to an outer cannula of a tracheal tube, inserting the inner cannula through a cover positioned over the first inlet, advancing the inner cannula through an outlet of the manifold into the outer cannula of the tracheal tube, and operating the manifold to rotate the first inlet from a first position that is traverse to the outlet of the manifold to a second position in which the first inlet is aligned with the outlet.

### BRIEF DESCRIPTION OF THE DRAWINGS

The detailed description particularly refers to the following figures, in which:

FIG. 1 is an exploded perspective view of a medical device system;

FIG. 2 is an exploded perspective view showing an inner cannula of the medical device system of FIG. 1;

FIG. 3 is a perspective view showing a distal end of the inner cannula positioned for insertion into a conduit of the medical device system of FIG. 1;

FIG. 4 is a cross-sectional perspective view of the conduit taken along the line 4-4 in FIG. 1;

FIG. 5 is a cross-sectional perspective view of the medical device system of FIG. 1 with the conduit in one operational position;

FIG. 6 is a perspective view of the conduit in another operational position; and

FIG. 7 is a cross-sectional perspective view taken along the line 7-7 in FIG. 6.

### DETAILED DESCRIPTION OF THE DRAWINGS

While the concepts of the present disclosure are susceptible to various modifications and alternative forms, specific exemplary embodiments thereof have been illustrated by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

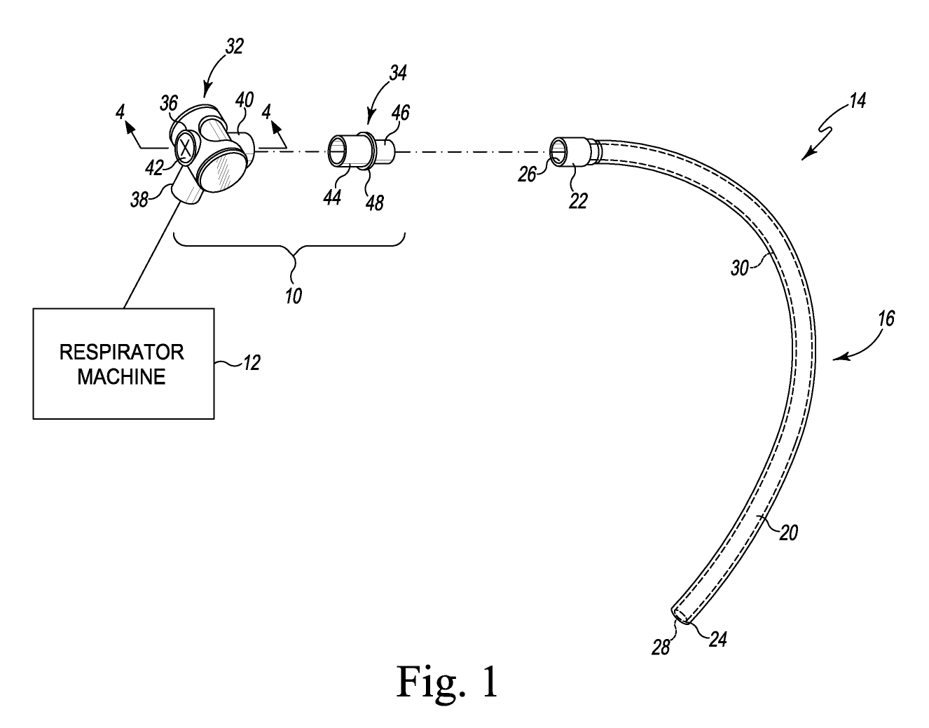

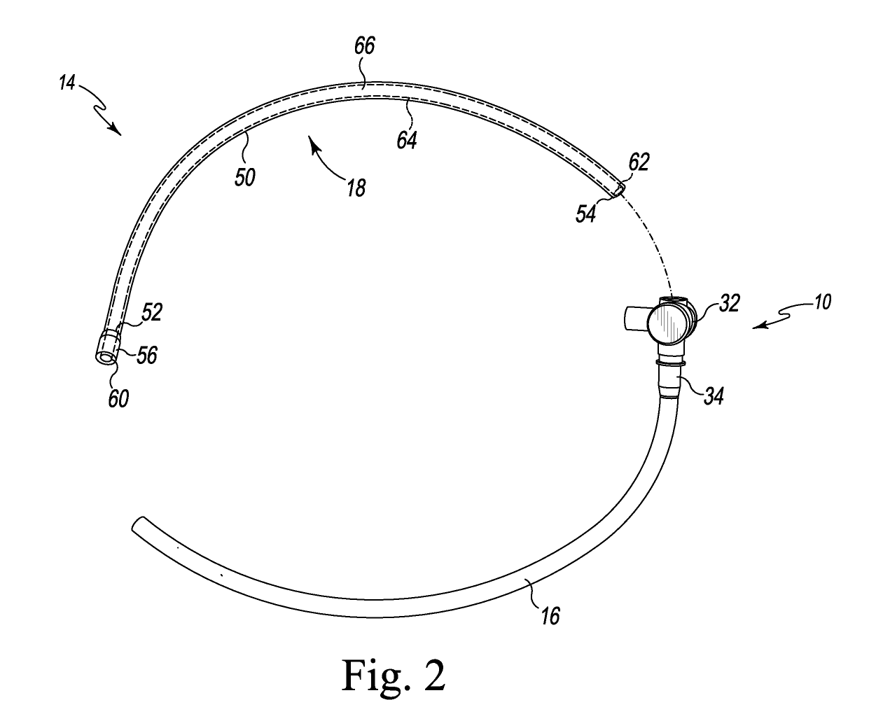

Referring now to FIG. 1, a medical device system including a conduit 10 configured to be connected to a respirator machine 12 and an endotracheal tube 14 is shown. In the illustrative embodiment, the tube 14 is an orotracheal tube configured for insertion through a patient's mouth into the patient's trachea. It should be appreciated that the structure and techniques described herein may be used with a nasotracheal tube or a tracheostomy tube. The endotracheal tube 14 includes an outer cannula 16 and an inner cannula 18 (see FIG. 2) configured to be inserted into the outer cannula 16.

The outer cannula 16 includes an elongated curved body 20 that extends from a proximal end 22 to a distal end 24. The curved body 20 has a circular opening 26 defined in the proximal end 22 and another circular opening 28 defined in the opposite distal end 24. A lumen 30 extends between the openings 26, 28 and has a curvature corresponding to the curvature of the body 20. In the illustrative embodiment, the lumen has a circular cross-section along its length. In other embodiments, the openings 26, 28 may be, for example, oval or oblong, and the lumen may have an oval or oblong cross-section. The cannula 16 is semi-rigid and formed from a biocompatible plastic resin such as, for example, polyvinyl carbonate, but it should be appreciated that in other embodiments other suitable materials may be used.

As shown in FIG. 1, the conduit 10 includes a manifold 32 and a connector 34 configured to connect the manifold 32 to the proximal end 22 of the outer cannula 16. As described in greater detail below, the manifold 32 includes a pair of inlets 36, 38 and an outlet 40 configured to be coupled to the connector 34. The inlet 36 is sized to receive the inner cannula 18 to permit the inner cannula 18 to be inserted into the outer cannula 16, and a cover 42 is positioned over the inlet 36. The other inlet 38 is configured to be connected to the respirator machine 12 via a connection tube, as shown in FIG. 1.

In the illustrative embodiment, the connector 34 includes a proximal sleeve 44 that is sized to be received in the outlet 40 of the manifold and a distal sleeve 46 that is sized to be received in the opening 26 defined in the proximal end 22 of the outer cannula 16. An annular rim 48 extends outwardly from the distal end of the proximal sleeve 44, which acts a stop to prevent over insertion of the connector 34 into the manifold 32 or cannula 16.

As shown in FIG. 2, the inner cannula 18 includes a body 50 that extends from a proximal end 52 to a distal end 54. The inner cannula 18 also includes a port 56 that extends proximally from the proximal end 52 of the body 50. The port 56 is also configured to be coupled to the respirator machine 12 via a connection tube (not shown).

The inner cannula 18 has a proximal opening 60 defined in the port 56 and an opposite distal opening 62 defined in the end 54 of the body 50. A passageway 64 extends between the openings 60, 62 and is sized to permit the passage of oxygen and other gases from a respirator machine 12 or other medical device attached to the port 56 downward into the patient's lungs. The passageway 64 has a circular cross-section along its length. In other embodiments, the openings 60, 62 may be, for example, oval or oblong, and the passageway may have an oval or oblong cross-section. In the illustrative embodiment, the body 50 and the port 56 are formed as a single semi-flexible monolithic component from a biocompatible plastic resin. In that way, the body 50 conforms to the shape of the curved lumen 30 of the outer cannula 16 when the body 50 is positioned in the lumen 30. In the illustrative embodiment, the body 50 of the cannula 18 also has a curved outer surface 66 that extends from the proximal end 52 to the distal end 54. It should be appreciated that the endotracheal tube 14 includes multiple inner cannulas 18 so that the inner cannula may be replaced as needed without replacement of the outer cannula 16.

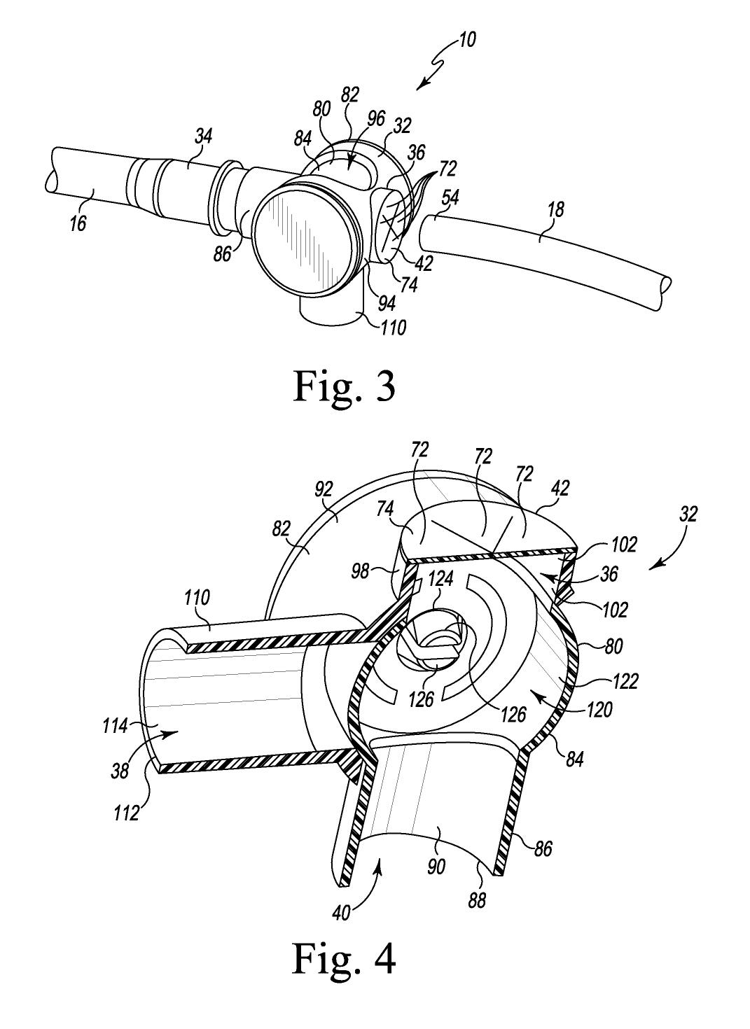

Referring now to FIG. 3, the distal end 54 of the inner cannula 18 is shown aligned with the inlet 36 of the conduit 10. In the illustrative embodiment, the conduit 10 includes a seal or cover 42 that closes the inlet 36. The seal 42 is structured, however, to open upon insertion of the inner cannula 18 to receive and allow it to be pushed down into the outer cannula 16 of the tube 14. The seal 42 is in the nature of a valve, which receives the inner cannula 18 and forms a substantial seal around it when installed in the opening of the manifold 32. Similarly, when the inner cannula 18 is removed all the way out of the respiratory path by complete removal from the opening of the inlet 36, the seal 42 is configured to reseal the inlet 36 to maintain the integrity of the closed respiration path. The seal 42 in the illustrative embodiment is automatically made upon insertion and removal of the inner cannula 18, without any further manipulation being necessary. Each is resiliently biased to close on itself or on a tube positioned through it.

In the illustrative embodiment, the seal 42 is formed from an elastomeric material such as, for example, silicone or rubber. As shown in FIGS. 3-4, the seal 42 includes a plurality of valve flaps 72 that extend from a circular rim 74 secured to the manifold 32. Each valve flap 72 is triangular-shaped, and the flaps 72 cooperate to define a cross-shaped slit in the seal 42. It should be appreciated that in other embodiments the seal 42 may include only a single valve flap 72 or additional flaps, and the flaps may take other geometric forms. In the illustrative embodiment, the flaps 72 are pressed inward when the cannula 18 is advanced into the manifold 32 and seal the inlet 36 by engaging the outer surface 66 of the cannula 18 and then the outer surface of the tube connecting the cannula 18 to the respirator machine 12. The flaps 72 are configured to resiliently move back to the position shown in FIG. 3 when the cannula 18 and/or respirator tube is removed.

The manifold 32 includes an inner component 80 and an outer component 82 that is pivotally coupled to the inner component 80. As shown in FIGS. 3-4, the inner component 80 includes a central body 84 and an outlet port 86 that extends outwardly from the central body 84. In the illustrative embodiment, the central body 84 is cylindrical. An opening 88 is defined in the port 86 and a cylindrical inner wall 90 extends inwardly from the opening 88 to define the outlet 40 of the manifold 32.

The outer component 82 includes a main body 92 that is positioned over the central body 84. The main body 92 has an outer cylindrical surface 94, and a transverse slot 96 that is defined in a section of the surface 94. As shown in FIG. 3, the slot 96 is sized to receive the port 86 of the inner component 80. Each of the components 80, 82 are formed separately from a biocompatible plastic resin such as, for example, polyvinyl carbonate, and are later assembled to form the manifold 32.

The outer component 82 includes a port 98 that extends outwardly from the main body 92. An opening 100 is defined in the port 98 and a cylindrical inner wall 102 extends inwardly from the opening 100 to define the inlet 36 of the manifold 32. As described above, the opening 100 is sized to receive the inner cannula 18. As shown in FIG. 4, the rim 74 of the cover 42 engages the port 98 to secure the cover 42 to the manifold 32. As described above, the inlet 36 is sized to receive the inner cannula 18. It should also be appreciated that the inlet 36 is sized to receive an endoscope sized to be inserted into the outer cannula 16 or a tool configured to clean the outer cannula. Additionally, in other embodiments, the inlet 36 may be sized to receive other tools or instruments to be inserted into the outer cannula 16.

The outer component 82 includes another port 110 that extends outwardly from the main body 92 traverse to the port 98. An opening 112 is defined in the port 110 and a cylindrical inner wall 114 extends inwardly from the opening 112 to define the inlet 38 of the manifold 32. As described above, the opening 112 is sized to receive the connection tube of the respirator machine 12 to connect the respirator machine to the conduit 10.

As shown in FIG. 4, the central body 84 of the inner component 80 includes a chamber 120 that selectively connects each of the inlets 36, 38 to the outlet 40. The chamber 120 is defined between a number of inner walls 122 of the inner component 80. In the illustrative embodiment, one of the inner walls 122 includes an opening 124 sized to receive resilient tabs 126 of the outer component 82, which are configured to be coupled to the outer component 82 to the inner component 80.

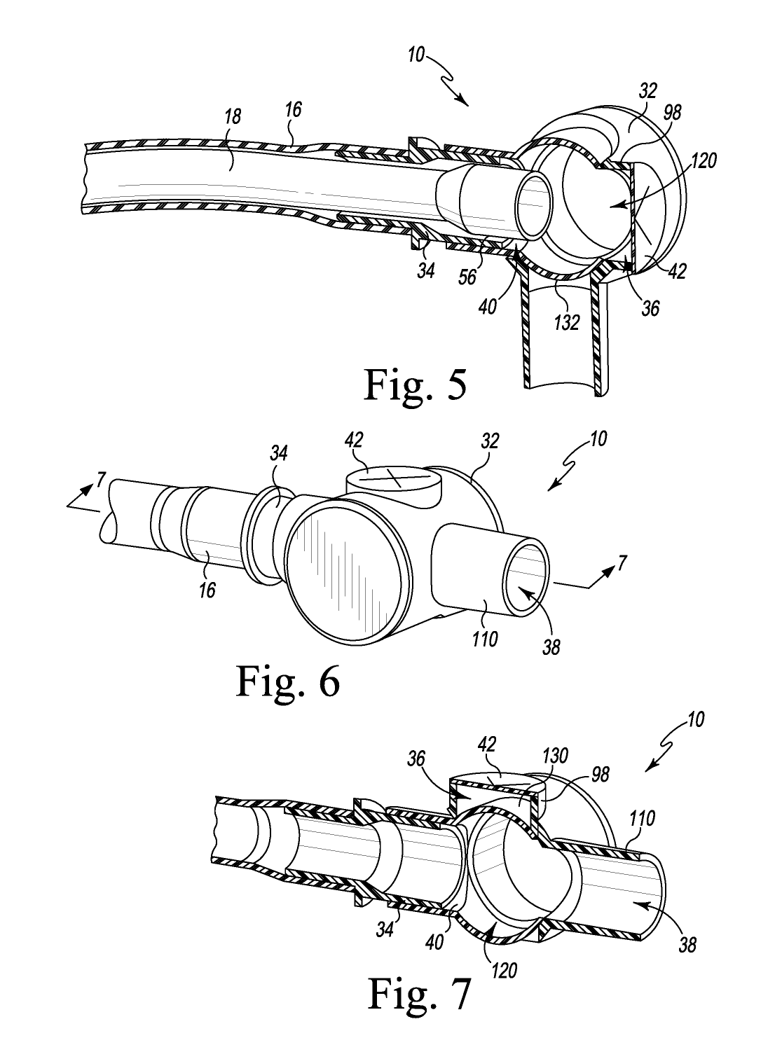

Referring now to FIG. 5, the inner cannula 18 may extend outwardly from the outer cannula 16 when the cannulas 16, 18 are assembled. The port 56 of the inner cannula 18 may be positioned in the passageway extending through the connector 34 and extend outwardly into the chamber 120 of the manifold 32.

In use, a medical professional or other user may insert an outer cannula 16 into a patient's mouth, down the patient's pharynx, and through the patient's glottis so that the distal opening 28 of the cannula 16 is positioned in the patient's trachea above the patient's carina (not shown). The user may then connect the conduit 10 with the manifold 32 in the respirator operational position shown in FIG. 6 to a respirator machine 12 and then to the outer cannula 16. As shown in FIG. 7, the inlet 38 is aligned with the outlet 40 of the manifold 32 when the manifold 32 is in the respirator operational position, thereby permitting the flow of air from the inlet 38 to the outlet 40. The inlet 36 extends traverse to the outlet 40 in the respirator operational position and is blocked by a section 130 of the inner component 80 of the manifold 32 to maintain the integrity of the closed respiration path.

When the medical professional or other user desires to insert an inner cannula 18 into the outer cannula 16, the medical professional may rotate the outer component 82 relative to the inner component 80 of the manifold 32. As the component 82 is rotated, the outlet port 86 of the outer component 82 is moved along the transverse slot 96 defined in the outer component 82 and the inlet 38 is moved out of alignment with the outlet 40. As shown in FIG. 5, the inlet 36 is moved into the alignment with the outlet 40 such that the inner cannula 18 may be advanced through the cover 42, into the inlet 36 and chamber 120 of the manifold 32, through the outlet 40 and connector 34, and into the outer cannula 16. In that position, the inlet 38 extends traverse to the outlet 40 and is blocked by a section 132 of the inner component 80 of the manifold 32. As described above, the flaps 72 are pressed inward when the cannula 18 is advanced into the manifold 32 and seal the inlet 36 by engaging the outer surface 66 of the cannula 18 and then the outer surface of the tube connecting the cannula 18 to the respirator machine 12 to maintain the integrity of the closed respiration path.

While the disclosure has been illustrated and described in detail in the drawings and foregoing description, such an illustration and description is to be considered as exemplary and not restrictive in character, it being understood that only illustrative embodiments have been illustrated and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

What is claimed is:

1. A medical device system, comprising:
a tracheal tube configured for insertion into an opening of a patient, comprising:
an outer cannula extending from a proximal end to a distal end configured to be positioned between in a patient's trachea, the outer cannula including a lumen extending from a first opening defined in the proximal end to a second opening defined in the distal end, and
an inner cannula removably positioned in the lumen of the outer cannula, the inner cannula having a proximal end that extends outwardly from the first opening of the outer cannula, a distal end positioned adjacent to the distal end of the outer cannula, and a passageway extending through the distal and proximal ends, and
a manifold removably coupled to the proximal end of the outer cannula, the manifold including an outlet connected to the first opening of the lumen of the outer cannula, a first inlet sized to receive and permit the passage of the inner cannula through the manifold into the first opening of the lumen of the outer cannula, and a second inlet configured to be coupled to an outlet of a respirator machine to permit airflow from the second inlet into the outlet, and
a cover positioned over the first inlet, the cover including at least one valve flap operable to be moved between a first position in which the first inlet is sealed and a second position in which the inner cannula is permitted to enter the first inlet.

2. The medical device system of claim 1, wherein:
the inner cannula includes a curved outer surface that extends between its distal end and its proximal end, and
the at least one flap is configured to engage the curved outer surface to create a seal between the inner cannula and the at least one valve flap when the inner cannula is positioned in the first inlet.

3. The medical device system of claim 1, wherein the cover and the at least one valve flap are formed from an elastomeric material.

4. The medical device system of claim 3, wherein the elastomeric material is at least one of rubber and silicone.

5. The medical device system of claim 1, wherein the at least one valve flap includes a plurality of triangular valve flaps extending from an outer rim of the cover, the outer rim being coupled to the manifold.

6. The medical device system of claim 1, wherein the manifold includes a first housing and a second housing movable coupled to the first housing.

7. The medical device system of claim 6, wherein the second housing is movable between a first position in which the first inlet is aligned with the outlet and a second position in which the first inlet is positioned transverse to the outlet.

8. The medical device system of claim 7, wherein when the second housing is the second position, a section of the first housing is positioned over the first inlet.

9. The medical device system of claim 7, wherein the second inlet is in fluid communication with the outlet when the second housing is in the second position.

10. The medical device system of claim 9, wherein the second inlet is positioned transverse to the outlet when the second housing is in the first position and the second inlet is aligned with the outlet when the second housing is in the second position.

11. The medical device system of claim 10, further comprising a connector having a first end sized to be positioned in the opening defined in the proximal end of the outer cannula and a second end sized to be positioned over the outlet of the manifold.

12. A medical device system, comprising:
a tracheal tube configured for insertion into an opening of a patient, the tracheal tube including an outer cannula,
a conduit configured to be coupled to a proximal end of the tracheal tube, the conduit including (i) a manifold comprising an outlet connected to the outer cannula, a first inlet sized to receive and permit the passage of an inner cannula through the manifold into the outer cannula, and a second inlet configured to be coupled to an outlet of a respirator machine to permit airflow from the second inlet into the tracheal tube, and (ii) a cover positioned over the first inlet, the cover including at least one valve flap operable to selectively seal the first inlet.

13. The medical device system of claim 12, wherein the inner cannula is one of a plurality of inner cannulas is sized to be positioned in the outer cannula.

14. The medical device system of claim 12, wherein the at least one flap includes a plurality of triangular flaps extending from an outer rim of the cover, the outer rim being coupled to the manifold.

15. The medical device system of claim 12, wherein the manifold includes a first housing and a second housing movable coupled to the first housing.

16. The medical device system of claim 15, wherein the second housing is movable between a first position in which the first inlet is aligned with the outlet and a second position in which the first inlet is positioned transverse to the outlet.

17. The medical device system of claim 16, wherein when the second housing is the second position, a section of the first housing is positioned over the first inlet.

18. The medical device system of claim 16, wherein the second inlet is in fluid communication with the outlet when the second housing is in the second position.

19. A method of operating a medical device system, comprising:
aligning an inner cannula with a first inlet of a manifold coupled to an outer cannula of a tracheal tube,
inserting the inner cannula through a cover positioned over the first inlet,
advancing the inner cannula through an outlet of the manifold into the outer cannula of the tracheal tube, and
operating the manifold to rotate the first inlet from a first position that is traverse to the outlet of the manifold to a second position in which the first inlet is aligned with the outlet.
